(19) United States
(12) Patent Application Publication
(10) Pub. No.: US 2026/0306185 A1
(43) Pub. Date: Oct. 8, 2026

(54) SURFACE-TRACKING SENSOR GRID AND METHOD FOR SHIELDING AN IN-VIVO INTERVENTIONAL SYSTEM FROM AN EXTERNAL MAGNETIC DISTORTION

(71) Applicant: Magnisity Ltd., Hod-HaSharon (IL)

(72) Inventors: Amit COHEN, Ramat Gan (IL); Ron BARAK, Tel-Aviv (IL); Benjamin GREENBURG, Hod-HaSharon (IL)

(73) Assignee: Magnisity Ltd., Hod-HaSharon (IL)

(21) Appl. No.: 19/481,277

(22) PCT Filed: May 2, 2024
(86) PCT No.: PCT/IL2024/050413

§ 371 Date: Nov. 4, 2025

Related U.S. Application Data
(60) Provisional application No. 63/463,903, filed on May 4, 2023.

(51) Int. Cl. A61B 34/20

(52) U.S. Cl. CPC A61B 34/20; A61B 2034/2051; A61B 2034/2061

(57) ABSTRACT

The invention discloses a system and method for shielding an in-vivo interventional system from an external electromagnetic (EM) distortion, the system including: at least one distortion shield having multiple magnetic field sensors; a distortion model of the distorting magnetic field produced by a distorting apparatus, a generated field model modeling the magnetic field generated by a transmitter; and a processing module, configured to: receive total magnetic field values measured by corresponding various sensors; construct, based on the received total measured fields, the distortion model, the generated field model, an energy cost function of the generated field and the distortion magnetic field possibly generated by the distorting apparatus; and determine modeled parameter values of the sensors, of the generated field and of the distortion magnetic field so that the energy cost function is minimized. The distortion shield may include a surface-tracking sensor grid.

### RELATED APPLICATION/S

This application claims the benefit of priority of U.S. Provisional Patent Application No. 63/463,903 filed on 4 May 2023, the contents of which are incorporated herein by reference in their entirety.

### FIELD AND BACKGROUND OF THE INVENTION

The present invention, in some embodiments thereof, relates to system and methods for shielding against distortions and, more particularly, but not exclusively, to a surface-tracking sensor grid and method for shielding an in-vivo interventional system from an external electromagnetic (EM) distortion.

An environment set for an interventional medical operation may include various instruments, some of them may include conductive and/or magnetically permeable components.

When location and/or shape sensing of an in-vivo interventional device depends on magnetic field measurements, the external instruments may generate a distorting magnetic field, in response to the generated tracking system's magnetic fields or intrinsic instrument's magnetic fields, that may interfere with the measurements and cause inaccurate location and/or shape calculations. The distorting magnetic field may be created due to eddy currents in the external instruments, as well as due to magnetization of ferromagnetic materials in the external instruments, or due to intrinsic electrical currents in the external instrument which may generate EM interference fields in frequencies which are similar or close to the tracking system operating frequencies.

Additional background art includes U.S. Pat. No. 11,486,958B2 disclosing a method and apparatus for reducing magnetic tracking error in the position and orientation determined in an electromagnetic tracking system. A corrected position and orientation is blended with an uncorrected position and orientation based upon the calculated probability of each. To determine a corrected position and orientation, data from an IMU in the receiver is used to obtain a constraint on the orientation. The amount of detected error due to electromagnetic distortion is measured. Any error is first assumed to be from “floor distortion,” and a correction is applied. If the error is still deemed too great, a constraint is again obtained from IMU data. Using this constraint, another correction for the distortion is made. The solution from this correction may be blended with a standard solution and the solution from the floor distortion to arrive at a final solution.

U.S. Pat. No. 11,944,388B2 disclosing navigation systems and methods for magnetic interference correction involve antennae that generate magnetic fields at different frequencies, sensors that measure the magnetic fields, and a computing device that uses sensor measurements to determine the magnetic interference and produce accurate sensor position and orientation information.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. A system for shielding an in-vivo interventional EM tracking system from an external magnetic distortion, the system comprising:
a. a plurality of magnetic field sensors;
b. a processing module;
c. a memory storing:
   i. a distortion model of a distorting magnetic field produced by one or more distorting apparatuses; and
   ii. a generated field model modeling the magnetic field generated by a transmitter; and
   iii. instructions to instruct said processing module for:
      A. receiving total magnetic field values measured by corresponding sensors from said plurality of magnetic field sensors; and
      B. determining modeled parameter values of said plurality of magnetic field sensors and of the distortion magnetic field by fitting said distortion model and said generated field model to said received total magnetic field values.

Example 2. The system according to example 1, wherein said instructions further comprise constructing an energy cost function of the generated field and the distortion magnetic field generated by the one or more distorting apparatuses; and wherein said fitting is so that the energy cost function is minimized.

Example 3. The system according to example 1 or example 2, wherein said plurality of magnetic field sensors are mounted on at least one distortion shield.

Example 4. The system according to any one of examples 1-3, wherein said at least one distortion shield is configured to be disposed between or in the proximity of a magnetic field transmitter and one or more distorting apparatuses.

Example 5. The system according to any one of examples 1-4, wherein said plurality of magnetic field sensors are mounted along at least one interventional elongated device.

Example 6. The system according to any one of examples 1-5, wherein said constructing an energy cost function is based on:
a. said received total measured fields;
b. said distortion model; and
c. said generated field model.

Example 7. The system according to any one of examples 1-6, wherein said distortion model is based on approximating said distorting magnetic fields as magnetic fields produced by one or more magnetic dipoles.

Example 8. The system according to any one of examples 1-7, wherein said distortion model is based on approximating said distorting magnetic fields as magnetic fields produced by one or more electrical current loops.

Example 9. The system according to any one of examples 1-8, wherein said distortion model is based on approximated magnetic permeability properties of said one or more distorting apparatuses.

Example 10. The system according to any one of examples 1-9, wherein said plurality of magnetic field sensors of said on at least one distortion shield are fixed to a body of a patient.

Example 11. The system according to any one of examples 1-10, wherein the processing module is configured to calculate motion parameter values of said body based on said measured magnetic fields.

Example 12. The system according to any one of examples 1-11, wherein said energy cost function is further constructed based on modeled motion parameters.

Example 13. The system according to any one of examples 1-12, wherein said modeled parameter values are further determined so that said energy cost function is minimized.

Example 14. The system according to any one of examples 1-13, wherein said plurality of magnetic field sensors of said on at least one distortion shield are combined with motion sensors for sensing motion of a body of a patient.

Example 15. The system according to any one of examples 1-14, wherein said determining modeled parameter values of said plurality of magnetic field sensors is made with relative positions and orientations of said plurality of magnetic field sensors as constraints.

Example 16. The system according to any one of examples 1-15, further comprising at least one interventional elongated device; and wherein said energy function comprises modeled parameters of said at least one interventional elongated device.

Example 17. The system according to any one of examples 1-16, wherein said energy function comprises smoothness and/or length constraints of said at least one interventional elongated device.

Example 18. The system according to any one of examples 1-17, wherein said energy function comprises sensed values of magnetic fields at multiple sensors along said at least one interventional elongated device; and wherein said instructions further comprise determining parameter values of said plurality of magnetic field sensors of said at least one interventional elongated device so that the cost function is minimized.

Example 19. The system according to any one of examples 1-18, wherein said instructions further comprise determining positions and orientations of said plurality of magnetic field sensors of said at least one interventional elongated device based on optimization approximated sensed values of magnetic fields.

Example 20. The system according to any one of examples 1-19, wherein said instructions further comprise receiving total magnetic field values measured by corresponding various sensors from said plurality of magnetic field sensors of said at least one interventional elongated device.

Example 21. The system according to any one of examples 1-20, wherein said instructions further comprise constructing said energy cost function further based on modeled parameters of said plurality of magnetic field sensors of said at least one interventional elongated device.

Example 22. The system according to any one of examples 1-21, wherein said instructions further comprise determining parameter values of said plurality of magnetic field sensors of said at least one interventional elongated device, so that the energy cost function is minimized.

Example 23. The system according to any one of examples 1-22, wherein said instructions further comprise determining a shape of said at least one interventional elongated device, based on optimization approximated sensed values of magnetic field.

Example 24. The system according to any one of examples 1-23, further comprising displaying a shape and/or position of said at least one interventional elongated device based on said determining.

Example 25. The system according to any one of examples 1-24, wherein said instructions further comprise determining positions and orientations of said plurality of magnetic field sensors of said on at least one distortion shield based on sensed values of magnetic fields.

Example 26. The system according to any one of examples 1-25, wherein said positions and orientations of said plurality of magnetic field sensors of said on at least one distortion shield are solved to determine and track dynamic positions and orientations of said at least one distortion shield.

Example 27. The system according to any one of examples 1-26, wherein said tracked positions and orientations of said plurality of magnetic field sensors of said on at least one distortion shield are used to track a surface of a body of a patient.

Example 28. The system according to any one of examples 1-27, wherein said tracked surface is used to model movements of said patient.

Example 29. The system according to any one of examples 1-28, wherein said modeled movements are used to update a real-time registration between tracked tools and an anatomy of said patient.

Example 30. The system according to any one of examples 1-29, wherein said tracked surface is used to track a deformation in a deformable registration model of an anatomy of said patient.

Example 31. The system according to any one of examples 1-30, wherein said tracked surface is used to track one or more of a breathing phase, an amplitude and a deformation caused by breathing to an anatomy of said patient.

Example 32. The system according to any one of examples 1-31, wherein said tracked surface is used to provide registration between said patient and a preoperative scan of said patient.

Example 33. The system according to any one of examples 1-32, wherein said provide a registration is done by segmenting a surface in said preoperative scan and matching said segmented surface to said tracked surface.

Example 34. The system according to any one of examples 1-33, wherein said tracked surface is used to improve shape and localization calculations by incorporating in said calculations movement of said patient with respect to a transmitter.

Example 35. The system according to any one of examples 1-34, wherein said at least one distortion shield is further configured for measuring at least one of temperature, heart rate, ECG, perspiration and respiration of a patient.

Example 36. A method for shielding an in-vivo interventional EM tracking system from an external magnetic distortion, comprising:
a. receiving total magnetic field values measured by corresponding sensors from a plurality of magnetic field sensors; and
b. determining modeled parameter values of said plurality of magnetic field sensors and of the distortion magnetic field by fitting said distortion model and said generated field model to said received total magnetic field values.

Example 37. The method according to example 36, further comprising constructing an energy cost function of the generated field and the distortion magnetic field generated by one or more distorting apparatuses; and wherein said fitting is so that the energy cost function is minimized.

Example 38. The method according to example 36 or example 37, further comprising mounting said plurality of magnetic field sensors on at least one distortion shield.

Example 39. The method according to any one of examples 36-38, further comprising disposing said at least one distortion shield between or in the proximity of a magnetic field transmitter and one or more distorting apparatuses.

Example 40. The method according to any one of examples 36-39, further comprising mounting said plurality of magnetic field sensors along at least one interventional elongated device.

Example 41. The method according to any one of examples 36-40, further comprising basing said constructing an energy cost function on:
a. said received total measured fields;
b. said distortion model; and
c. said generated field model.

Example 42. The method according to any one of examples 36-41, further comprising basing said distortion model on approximating said distorting magnetic fields as magnetic fields produced by one or more magnetic dipoles.

Example 43. The method according to any one of examples 36-42, further comprising basing said distortion model on approximating said distorting magnetic fields as magnetic fields produced by one or more electrical current loops.

Example 44. The method according to any one of examples 36-43, further comprising basing said distortion model on approximated magnetic permeability properties of said one or more distorting apparatuses.

Example 45. The method according to any one of examples 36-44, further comprising fixing said plurality of magnetic field sensors of said on at least one distortion shield to a body of a patient.

Example 46. The method according to any one of examples 36-45, further comprising calculating motion parameter values of said body based on said measured magnetic fields.

Example 47. The method according to any one of examples 36-46, further comprising further constructing said energy cost function based on modeled motion parameters.

Example 48. The method according to any one of examples 36-47, further comprising further determining said modeled parameter values so that said energy cost function is minimized.

Example 49. The method according to any one of examples 36-48, further comprising combining said plurality of magnetic field sensors of said on at least one distortion shield with motion sensors for sensing motion of a body of a patient.

Example 50. The method according to any one of examples 36-49, wherein said determining modeled parameter values of said plurality of magnetic field sensors is made with relative positions and orientations of said plurality of magnetic field sensors as constraints.

Example 51. The method according to any one of examples 36-50, wherein said energy function comprises modeled parameters of at least one interventional elongated device.

Example 52. The method according to any one of examples 36-51, wherein said energy function comprises smoothness and/or length constraints of said at least one interventional elongated device.

Example 53. The method according to any one of examples 36-52, wherein said energy function comprises sensed values of magnetic fields at multiple sensors along said at least one interventional elongated device; and further comprising determining parameter values of said plurality of magnetic field sensors of said at least one interventional elongated device so that the cost function is minimized.

Example 54. The method according to any one of examples 36-53, further comprising determining positions and orientations of said plurality of magnetic field sensors of said at least one interventional elongated device based on optimization approximated sensed values of magnetic fields.

Example 55. The method according to any one of examples 36-54, further comprising receiving total magnetic field values measured by corresponding various sensors from said plurality of magnetic field sensors of said at least one interventional elongated device.

Example 56. The method according to any one of examples 36-55, further comprising constructing said energy cost function further based on modeled parameters of said plurality of magnetic field sensors of said at least one interventional elongated device.

Example 57. The method according to any one of examples 36-56, further comprising determining parameter values of said plurality of magnetic field sensors of said at least one interventional elongated device, so that the energy cost function is minimized.

Example 58. The method according to any one of examples 36-57, further comprising determining a shape of said at least one interventional elongated device, based on optimization approximated sensed values of magnetic field.

Example 59. The method according to any one of examples 36-58, further comprising displaying a shape and/or position of said at least one interventional elongated device based on said determining.

Example 60. The method according to any one of examples 36-59, further comprising determining positions and orientations of said plurality of magnetic field sensors of said on at least one distortion shield based on sensed values of magnetic fields.

Example 61. The method according to any one of examples 36-60, further comprising solving said positions and orientations of said plurality of magnetic field sensors of said on at least one distortion shield for determining and tracking dynamic positions and orientations of said at least one distortion shield.

Example 62. The method according to any one of examples 36-61, further comprising using said tracked positions and orientations of said plurality of magnetic field sensors of said on at least one distortion shield for tracking a surface of a body of a patient.

Example 63. The method according to any one of examples 36-62, further comprising using said tracked surface for modeling movements of said patient.

Example 64. The method according to any one of examples 36-63, further comprising using said modeled movements for updating a real-time registration between tracked tools and an anatomy of said patient.

Example 65. The method according to any one of examples 36-64, further comprising using said tracked surface for tracking a deformation in a deformable registration model of an anatomy of said patient.

Example 66. The method according to any one of examples 36-65, further comprising using said tracked surface for tracking one or more of a breathing phase, an amplitude and a deformation caused by breathing to an anatomy of said patient.

Example 67. The method according to any one of examples 36-66, further comprising using said tracked surface for providing registration between said patient and a preoperative scan of said patient.

Example 68. The method according to any one of examples 36-67, further comprising segmenting a surface in said preoperative scan and matching said segmented surface to said tracked surface.

Example 69. The method according to any one of examples 36-68, further comprising using said tracked surface for improving shape and localization calculations by incorporating in said calculations movement of said patient with respect to a transmitter.

Example 70. The method according to any one of examples 36-70, further comprising measuring at least one of temperature, heart rate, ECG, perspiration and respiration of a patient using said at least one distortion shield.

Example 71. A method for shielding an in-vivo interventional EM tracking system from an external magnetic distortion, comprising:
a. constructing an energy cost function;
b. minimizing said energy cost function thereby finding location of sensors and finding distortion parameters;
d. solving a location of said sensors.

Example 72. The method according to example 71, further comprising predicting distortion fields at any location in 3D space.

Example 73. The method according to example 71 or example 72, further comprising assuming one or more of:
a. that a distortion field can be modeled;
b. that sensors read certain magnetic field measurements; and
c. that sensors are located at unknown positions and orientations.

Example 74. The method according to any one of examples 71-73, wherein said solving a location of said sensors comprises constructing a second energy cost function using said found distortion parameters.

Example 75. The method according to any one of examples 71-74, further comprising minimizing said second energy cost function.

According to an aspect of some embodiments of the present invention there is provided a system for shielding an in-vivo interventional system from an external magnetic distortion, the system comprising:
at least one distortion shield configured to be disposed between or in the proximity of a magnetic field transmitter and one or more distorting apparatus, the shield having multiple magnetic field sensors;
a memory storing a distortion model of the distorting magnetic field produced by the distorting apparatus, and a generated field model modeling the magnetic field generated by the transmitter; and a processing module, wherein the memory further stores instructions, configured to instruct the processing module to:
receive total magnetic field values measured by corresponding various shield sensors;
construct, based on the received total measured fields, the distortion model, and the generated field model, an energy cost function of the generated field and the distortion magnetic field possibly generated by the distorting apparatus; and
determine modeled parameter values of the shield sensors, of the generated field and of the distortion magnetic field so that the energy cost function is minimized.

According to some embodiments of the invention, the distortion model is based on approximating the distorting magnetic fields as magnetic fields produced by one or more magnetic dipoles.

According to some embodiments of the invention, the distortion model is based on approximating the distorting magnetic fields as magnetic fields produced by one or more electrical current loops.

According to some embodiments of the invention, the distortion model is based on approximated magnetic permeability properties of the distorting apparatus.

According to some embodiments of the invention, the distortion shield sensors are fixed to a body of a patient, wherein the processing module is configured to calculate motion parameter values of the body based on the measured magnetic fields.

According to some embodiments of the invention, the energy cost function is further constructed based on modeled motion parameters, and modeled parameter values are further determined so that the energy cost function is minimized.

According to some embodiments of the invention, the shield sensors are combined with motion sensors for sensing motion of the body.

According to some embodiments of the invention, the optimization may be made with relative positions and orientations of the various sensors as constraints.

According to some embodiments of the invention, the system comprises at least one tracked elongated flexible device, wherein the energy function comprises modeled parameters of the at least one tracked elongated flexible device.

According to some embodiments of the invention, the energy function comprises smoothness and/or length constraints of the at least one tracked device.

According to some embodiments of the invention, the energy function comprises sensed values of magnetic field at multiple sensors along an elongated flexible device, and the instructions are configured to instruct the processing module to determine parameter values of the device sensors so that the cost function is minimized.

According to some embodiments of the invention, the instructions are configured to instruct the processing module to determine positions and orientations of the device sensors based on optimization approximated sensed values of magnetic field.

According to some embodiments of the invention, the instructions are configured to instruct the processing module to receive total magnetic field values measured by corresponding various device sensors; construct the energy cost function further based on modeled parameters of the various device sensors; and further determine parameter values of the device sensors, so that the energy cost function is minimized.

According to some embodiments of the invention, the instructions are configured to instruct the processing module to determine a shape of the at least one tracked elongated device, based on optimization approximated sensed values of magnetic field.

According to some embodiments of the invention, the instructions are configured to instruct the processing module to determine positions and orientations of the distortion shield magnetic sensors based on sensed values of magnetic field.

According to some embodiments of the invention, positions and orientations of the distortion shield magnetic sensors are solved to determine and track dynamic positions and orientations of the distortion shield grid.

According to some embodiments of the invention, the tracked positions and orientations of the distortion shield grid sensors are used to track a surface of the patient's body.

According to some embodiments of the invention, the tracked surface of the patient's body is used in the procedure to model the patient's movements, to update a real-time registration between tracked tools and the patient's anatomy.

According to some embodiments of the invention, the tracked surface of the patient's body is used to track the deformation in a deformable registration model of the patient's anatomy.

According to some embodiments of the invention, the tracked surface of the patient's body is used to track the breathing phase and amplitude and the deformation caused by the breathing to the patient's anatomy.

According to some embodiments of the invention, the tracked surface of the patient's body is used to provide registration between the patient and a preoperative scan of the patient, by segmenting the patient's surface in the preoperative scan and matching it to the real-time tracked surface of the patient's body.

According to some embodiments of the invention, the tracked surface of the patient's body is used to improve shape and localization calculations by incorporating in the calculation the patient's movement with respect to the transmitter.

According to some embodiments of the invention, the distortion shield measures at least one of the patient's temperature, heart rate, ECG, perspiration, respiration.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1a and 1b, showing a schematic representation of an exemplary interventional system and an exemplary operational setting, according to some embodiments of the invention; and

FIG. 2 is a flowchart of an exemplary method for shielding an in-vivo interventional system from an external magnetic distortion, according to some embodiments of the invention.

### DESCRIPTION OF SPECIFIC EMBODIMENTS OF THE INVENTION

The present invention, in some embodiments thereof, relates to system and methods for shielding against distortions and, more particularly, but not exclusively, to a surface-tracking sensor grid and method for shielding an in-vivo interventional system from an external electromagnetic (EM) distortion.

### Overview

An aspect of some embodiments of the invention relates to a system configured to shield and/or compensate against external magnetic distortions in an in-vivo interventional system. The terms “shield” and “shielding” refer to means and methods to compensate for distortions to the electromagnetic field as sensed by the system caused by external causes and/or means and methods to avoid and/or cancel the effects of distortions to the electromagnetic field caused by external causes as sensed by the system. In some embodiments, the system comprises a plurality of sensors (on an interventional device being tracked and/or a grid—see below) configured for sensing generated electromagnetic (EM) fields and dedicated software having instructions to perform actions that enable the system to differentiate between the generated EM fields and the distorting EM fields.

In some embodiments, magnetic tracking is used to track the location and/or the shape and/or tip location of an interventional device within a patient, where one or more EM fields are generated outside the patient and one or more sensors are positioned along the interventional device, which is being used within the patient. In some embodiments, the sensors are configured for sensing known (pre-modeled) magnetic fields generated by an external transmitter. In some embodiments, each of the sensors of the interventional device measures the total magnetic fields at their location and orientation. In some embodiments, while the transmitter generates known magnetic fields at each point in space, additional nearby objects may affect the magnetic fields. In some embodiments, as will be further explained below, the system is configured to use the data from the sensed EM field by the sensors on the interventional device and differentiate between the generated EM fields and possible distorting EM fields.

In some embodiments, the system comprises a grid of sensors attached to the body of the patient. In some embodiments, the grid of sensors is used to shield for metal distortions in the setting of magnetic tracking. In some embodiments, the sensors in the grid are tracked and used to sense and separate between the intentionally generated magnetic fields, which are used for example for tracking, to superimposed fields of a magnetic distorter. In some embodiments, the separation between the transmitter's fields and the distortion fields may be done based on one or more models, for example a physical modeling of the distortion fields, and the accurate measurements of the sensors. In some embodiments, optionally, the sensors are constrained by known geometrical constraints such as relative positions and orientations of the sensors as being placed on the body of the user (or the interventional device as explained above).

In some embodiments, the sensors of the interventional device are “shielded” from distortions to the transmitter's magnetic fields, for example, by the array of sensors positioned between or in the proximity of the interventional device's sensors and a distorting apparatus.

An aspect of some embodiments of the invention relates to system and methods for compensating for one or more external magnetic distortions in an in-vivo interventional system, where the system comprises one or more of:
1. At least one distortion shield configured to be disposed between or in the proximity of a magnetic field transmitter and a distorting apparatus, the shield comprising multiple magnetic field sensors;
2. A memory storing:
   a. A distortion model of the distorting magnetic field produced by the one or more distorting apparatus, and
   b. A generated field model modeling the magnetic field generated by the transmitter; and
3. A processing module.

In some embodiments, the memory further stores instructions for instructing the processing module for:
1. Receiving total magnetic field values measured by corresponding various shield sensors;
2. Constructing, based on:
   a. The received total measured fields;
   b. The distortion model; and
   c. The generated field model;
   an energy cost function of the generated field and the distortion magnetic field possibly generated by the distorting apparatus; and
3. Determining modeled parameter values of the shield sensors, of the generated field and of the distortion magnetic field so that an energy cost function is minimized.

In some embodiments, the distortion model is based on approximating the distorting magnetic field as a magnetic field produced by one or more magnetic dipoles.

In some embodiments, the distortion model is based on approximating the distorting magnetic field as a magnetic field produced by one or more electrical current loops.

In some embodiments, the distortion model is based on approximated magnetic permeability properties of the distorting apparatus.

In some embodiments, some parameters of the distortion model are known in advance, where the known parameters are used to accelerate the convergence or to improve the accuracy of the optimization process during the determining modeled parameter values step (for example, and not limited to, reaching an energy cost function that is minimized).

In some embodiments, the distortion shield sensors are fixed to a patient's body, where the processing module is configured to calculate motion parameter values of the body based on the measured magnetic fields.

In some embodiments, the energy cost function is further constructed based on modeled motion parameters, and modeled parameter values are further determined so that the energy cost function is minimized.

In some embodiments, the shield sensors are combined with motion sensors (such as accelerometer, gyroscope or others) for sensing motion of the body, and wherein the energy cost function is further constructed based on modeled parameters of the motion sensors, and modeled parameter values of the motion sensors are further determined so that the energy cost function is minimized.

In some embodiments, the optimization may be made with relative positions and orientations of the various sensors as constraints.

In some embodiments, the energy function comprises smoothness and/or length constraints of the shield sensor arrays used in the optimization process.

In some embodiments, the energy function comprises modeled parameters of the at least one tracked elongated flexible device.

In some embodiments, the energy function comprises smoothness and/or length constraints of at least one tracked device used in the optimization process.

In some embodiments, the energy function comprises sensed values of magnetic field at multiple sensors along the elongated flexible device, and the instructions are configured to instruct the processing module to determine parameter values of the device sensors so that the cost function is minimized.

In some embodiments, the instructions comprise determining positions and orientations of the interventional device sensors and/or the grid sensors based on optimization approximated sensed values of magnetic field.

In some embodiments, the instructions comprise receiving total magnetic field values measured by corresponding various sensors (interventional device and/or grid); constructing the energy cost function further based on modeled parameters of the various sensors; and further determining parameter values of the sensors, so that the energy cost function is minimized.

In some embodiments, the instructions comprise determining a shape of the at least one tracked interventional elongated device, based on optimization approximated sensed values of magnetic field.

In some embodiments, the instructions comprise determining positions and orientations of the distortion shield magnetic sensors based on sensed values of magnetic field.

In some embodiments, positions and orientations of the distortion shield magnetic sensors are solved to determine and track dynamic positions and orientations of the distortion shield grid.

In some embodiments, the tracked positions and orientations of the distortion shield sensors are used to track a surface of the patient's body.

In some embodiments, the tracked surface of the patient's body is used in the procedure to model the patient's movements, for example, to update a real-time registration between tracked interventional tools and the patient's anatomy.

In some embodiments, the tracked surface of the patient's body is used to track the deformation in a deformable registration model of the patient's anatomy.

In some embodiments, the tracked surface of the patient's body is used to track the breathing phase and amplitude and the deformation caused by the breathing to the patient's anatomy.

In some embodiments, the tracked surface of the patient's body is used to provide registration between the patient and a preoperative scan of the patient, for example, by segmenting the patient's surface in the preoperative scan and matching it to the real-time tracked surface of the patient's body.

In some embodiments, the tracked surface of the patient's body is used to improve shape and localization calculations of the interventional device by incorporating in the calculation the patient's movement with respect to the transmitter. In this case, the interventional device may be assumed to move in correlation with the patient's body, such that if the patient's body is static, it is not expected that the interventional device will shift its global position in space, and vice versa—if the patient's body moves, for example, 10 cm to the right (along the transmitter's X axis) then the interventional device is also expected to move globally 10 cm to the right, in correlation with the patient's body. In some embodiments, by tracking a surface of the patient's body it is thus possible to compare between patient's body movements to interventional device movements and thus distinguish between true movements (for example, due to interventional device actually being manipulated or moved with body) to false movements (for example, due to bringing a dynamically positioned distorter apparatus into the tracking space, which may result in device's false movement due to EM distortion).

In some embodiments, the distortion shield measures at least one of the patient's temperature, heart rate, ECG, perspiration, respiration. For example, the distortion shield can be used to measure respiration by tracking the distortion shield position and extracting breathing pattern from the tracked surface. For example, a high-pass or band-pass filter can be used, for example, which filters out tracked surface motion frequencies which are not breathing frequencies, for example, lower than 10 per minute, or higher than 20 per minute. In some embodiments, the filtered track surface motion pattern can then be analyzed to extract breathing phase and amplitude using signal processing methods. In another embodiment, patient's heart rate can be extracted from the tracked surface motion by filtering frequencies which are lower than 50 beats per minute or higher than 200 beats per minute and employing signal processing methods to extract the heart beat pattern in the filtered track surface motion. In some embodiments, these methods make use of the existing magnetic field sensors on the distortion shield which provide surface tracking of the distortion shield to extract respiration and heart rate from the tracked surface motion. In some embodiments, with additional sensors more patient information can be measured. For example, by employing ECG sensors on the distortion shield, patient ECG can be performed. By employing temperature sensors, patient's body temperature can be tracked. In some embodiments, the magnetic field sensors are also temperature sensors (for example, in the case of some DC magnetometers). In some embodiments, the distortion shield can serve both as an EM distortion shield, as described herein, as well as an ECG patch which is usually necessary during various clinical procedures. In some embodiments, this potentially saves the need for an additional ECG patch and may combines a single sticker/patch for multiple purposes—for example, for tracking patient's body position, for modeling patient's organ deformation (by tracking the patient's body surface), for shielding against external EM distortion, for tracking patient's respiration, for tracking patient's body temperature, and for performing ECG of the patient.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

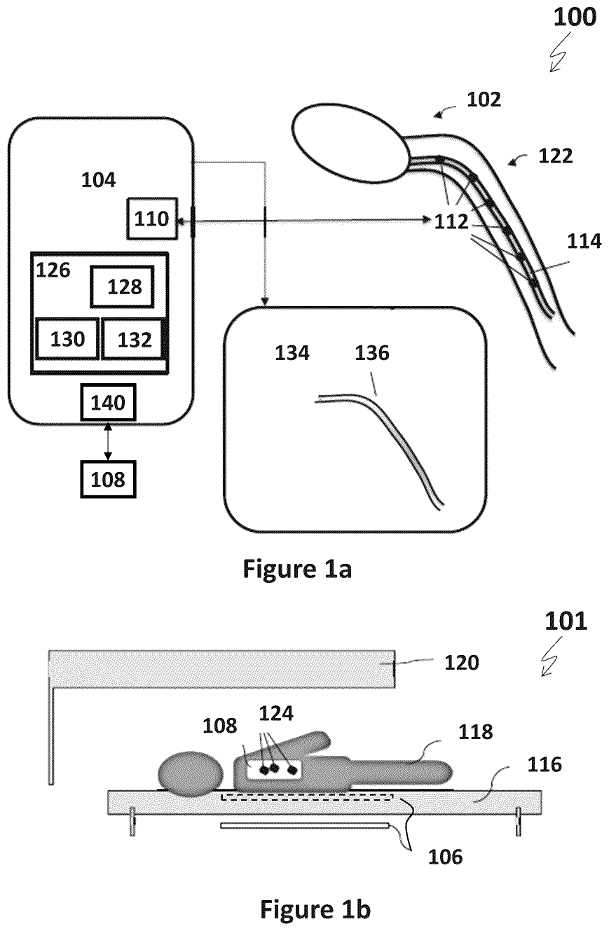

Referring now to FIGS. 1a and 1b, showing a schematic representation of an exemplary interventional system 100 and an exemplary operational setting 101, according to some embodiments of the invention.

In some embodiments, an exemplary interventional system 100 comprises one or more of: an interventional device 102, a processing module 104, a magnetic field transmitter 106 (which may be a flat magnetic field transmitter that can be placed below the patient's mattress or the patient's bed), and optionally a magnetic distortion shield 108 comprising a plurality of sensors 124. In some embodiments, the magnetic distortion shield 108 may be a disposable sticker/patch.

In some embodiments, magnetic distortion shield 108 is connected to an electromagnetic shape and/or position and/or surface tracking module 140, configured to sense the shape and/or position and/or surface of the distortion shield 108, based on magnetic field sensor readings, caused by one or more magnetic fields generated by transmitter 106 (shown in FIG. 1b).

In some embodiments, the electromagnetic shape and/or position and/or surface tracking module 140 is in communication with and/or receives information from a plurality of electromagnetic sensors 124 positioned on a surface of the distortion shield 108 and is configured to calculate a shape and/or position and/or surface of the distortion shield 108 accordingly.

In some embodiments, the electromagnetic shape and/or position and/or surface tracking module 140 is connected to processor module 104 and communicates one or more of: the magnetic field sensor readings of electromagnetic sensors 124; the calculated shape and/or position and/or surface of the distortion shield 108. In some embodiments, the communication is wired (for example, via USB). In some embodiments, the communication is wireless (for example, via WiFi, Bluetooth, etc.).

In some embodiments, the exemplary interventional system 100 further comprises an electromagnetic shape and/or position tracking module 110, configured to sense the shape and/or position of the interventional device 102, based on magnetic field sensor readings, caused by one or more magnetic fields generated by transmitter 106 (shown in FIG. 1b).

In some embodiments, the electromagnetic shape and/or position tracking module 110 is in communication with and/or receives information from a plurality of electromagnetic sensors 112 positioned along a length of a flexible elongated portion 114 of the interventional device 102 and is configured to calculate a shape and/or position of the interventional device 102 accordingly.

In some embodiments, the electromagnetic shape and/or position tracking module 110 is connected to processor module 104 and communicates one or more of: the magnetic field sensor readings of electromagnetic sensors 112, the calculated shape and/or position of interventional device 102. In some embodiments, the communication is wired (for example, via USB). In some embodiments, the communication is wireless (for example, via WiFi, Bluetooth, etc.).

In some embodiments, tracking modules 110 and 140 perform the tracking calculations independently. In this case they may communicate only the computed tracking results (shape, location, surface etc.) to processor module 104. In other embodiments, tracking modules 110 and 140 only sample the electromagnetic sensors 124 or 112, as well as additional sensors (if present). In this case they may communicate the sensor measurements to processor module 104, and the processor would then perform the tracking calculations, as described herein.

In some embodiments, the electromagnetic sensors 112 (as well as sensors 124) are one or more of EM coil-based sensors, digital magnetometer sensors, DC magnetometer sensors, hall-effect sensors, magneto-resistive sensors, magneto-inductive sensors, or any other sensors which are suitable for measuring magnetic fields. In some embodiments, optionally, the processing module 104 displays on a display 134, a representation 136 of the interventional device 102 with the calculated shape and/or position.

In some embodiments, in order to calculate the shape and/or position of the interventional device 102, the electromagnetic shape and/or position tracking module 110 calculates a plurality of locations along the interventional device 102, for example relative to the transmitter 106, based on known generated magnetic fields at the transmitter 106 and the magnetic fields sensed by the sensors 112 along the interventional device 102. In some embodiments, optionally, a plurality of sensors 112 communicate the sensed local magnetic fields utilizing a mutual digital bus 114.

In some embodiments, during operation, the system 100 is optionally installed utilizing a setting 101 as shown for example in FIG. 1b, which is suitable for an interventional operation. In some embodiments, setting 101 comprises one or more of: a table (or patient bed) 116, on which a patient 118 may be positioned. In some embodiments, setting 101 may further include one or more distorting apparatuses 120 (one shown in FIG. 1b), which may, for example, consist of one or more of an imaging device such as CT, CBCT (Cone-beam CT) or C-arm fluoroscopic device, an illumination device, a robotic device, a display device, a computing device, an operational device, a medical device, and any other suitable kind of device. In some embodiments, during operation, the transmitter 106 may be installed under patient 118, for example under the table (or patient bed) 116, or above the patient bed 116 and under the patient's mattress. In some embodiments, the flexible elongated portion 122 of the interventional device 102 is inserted into an anatomy of patient 118, and the system is configured for tracking a location and/or a shape of the flexible elongated portion 122 of the interventional device 102, while being inside the patient.

The one or more distorting apparatuses 120 may include magnetically permeable components, for example diamagnetic components and/or parts, which may be magnetized in response to the magnetic fields generated by the transmitter 106. Thus, the one or more distorting apparatuses 120 may in turn generate distorting magnetic fields, which may affect the sensed magnetic fields along the interventional device 102, thereby possibly distorting the sensed position and/or shape by the electromagnetic shape sensing module 110. The same may happen due to conductive components in the one or more distorting apparatuses 120 and eddy currents generated in the conductive components due to the fields generated by transmitter 106. For example, sensors 112 may sense distorted magnetic fields, which may be resulted from both the generated magnetic fields generated by transmitter 106 and those generated by the distorting apparatus, as a result of being magnetized by the generated magnetic fields generated by transmitter 106, or due to eddy currents generated in the conductive components due to the fields generated by transmitter 106.

In some embodiments, the system is configured to calculate, for example, by processor 104, the distortion component in the distorted magnetic fields (distorted magnetic fields=generated magnetic fields+the distorting magnetic fields). In some embodiments, the system is configured to perform these calculations using: 1. The data sensed by the sensors 112 in the interventional device 102; 2. The data sensed by the sensors 124 in the magnetic distortion shield 108; or 3. Using both the data sensed by the sensors 112 and the sensors 124.

### Exemplary Distortion Shield 108

In some embodiments, the distortion shield 108 comprises a plurality of magnetic field sensors 124, configured to be installed before or during operation between or in the proximity of the interventional device 102 and the one or more distorting apparatuses 120. In some embodiments, the magnetic field sensors 124 are installed/positioned on the body of patient 118, for example on the chest of the patient, using for example one or more of stickers/patch, a wearable garment and/or any other suitable manner/device. In some embodiments, the distortion shield is disposable, for example, a disposable sticker/patch. In some embodiments, the plurality of magnetic field sensors 124 are installed on an external holder (not shown), which facilitates the position of the sensors 124 between or in the proximity of the patient 118 and the one or more distorting apparatuses 120.

In some embodiments, the plurality of magnetic field sensors 124 are fixed to the body of patient 118 and/or are combined with other sensors. For example, sensors for sensing motion and/or momentary positions of the body, for example due to breathing of patient 118.

### Exemplary Calculation of the Distorted Component in the Sensed Distorted Magnetic Field

In some embodiments, the processing module 104 comprises instructions to subtract the distorting magnetic fields from the distorted magnetic fields, leaving only the generated magnetic fields, thus nullifying the distortion and enabling accurate sensing of the location and/or shape of the interventional device 102 by the electromagnetic shape-sensing module 110.

In some embodiments, the processing module 104 comprises one or more of a memory 126, storing therein instructions 128, to instruct the processing module 104 to perform operations of the methods described herein. In some embodiments, the memory 126 is configured to store one or more of:
1. A distortion model 130, modeling the form of the distortion magnetic field as generated by one or more distorting apparatuses 120; and
2. A generated field model 132, modeling the magnetic field as generated by transmitter 106.

In some embodiments, the processing module 104 uses models 130 and 132 to calculate the distortion component of the total, distorted, magnetic fields measured by sensors 124 and/or 112.

In some embodiments, the processing module 104 comprises instructions to perform one or more of:
1. Determining the value of the magnetic fields generated by the transmitter 106;
2. Computing the location (position and/or orientation) of the sensors 124 based on the found value of the magnetic field generated by transmitter 106;
3. Computing the location of the sensors 112 based on the found value of the magnetic field generated by transmitter 106.
   Exemplary Use of Generated Models to Subtract the Distorting Magnetic Fields from the Distorted Magnetic Fields

In some embodiments, the distorting magnetic fields sensed by the sensors (112 and/or 124) are modeled, for example and not limited to, as one or more magnetic dipole fields which origin at a certain location in 3D space and have certain one or more strength/amplitudes, and incorporated in an error function, for example together with data regarding the positions and orientations of the sensors 112 of the interventional device 102 and/or of the sensors 124 sensing the distorted magnetic fields.

In some embodiments, the error function is minimized by finding optimal parameter values of the distortion and of the sensors, thus determining the six degrees-of-freedom (6-DOF) sensor location and/or shape of the interventional device 102 and/or of distortion shield 108. For example, the optimal parameter values are those that minimize the error function. In case that the error function potentially has several local minimum points, optimal parameter values would then be those that minimize the error function globally. The error function can be minimized in an iterative non-linear optimization process such as Gradient Descent, Levenberg-Marquardt or any other suitable optimization method.

In some embodiments, optionally, the distortion fields (the EM fields that distort the generated EM fields) may also be solved under the same optimization process. In some embodiments, optionally, the distorter's position, orientation and strength may also be solved (according to some distortion model) under the same optimization process (see below).

In some embodiments, for example, the 6-DOF positions and orientations of the sensors are determined by including the position and orientation parameters of the sensors in an error function and solving for the optimal position and orientation parameter values.

In some embodiments, the distortion model 130 is based on approximating the distorting magnetic field as a magnetic field produced by one or more magnetic dipoles, or by one or more electrical current loops of certain orientation, diameter and/or shape (which can also be parametrized).

In some embodiments, the distortion model 130 may be based on the approximated magnetic permeability properties, for example the approximated diamagnetic properties of distorting apparatus 120.

In some embodiments, generated field model 132 is also approximated as a magnetic field produced by one or more magnetic dipoles, wherein the value of the generated magnetic field at transmitter 106 is known.

In some embodiments, other kinds of approximations and/or modeling may be used.

In some embodiments, the transmitter 106 may transmit a plurality of magnetic fields, for example by multiple transmitting electromagnetic coils, for example at corresponding different frequencies, for example generating magnetic fields of different geometries. In some embodiments, positions and/or orientations of magnetic sensors at various locations may be calculated by demultiplexing the sensed magnetic field over time into a plurality of sensed fields of different frequencies, for example by discrete Fourier transform, by correlation methods or by any other suitable method. In some embodiments, the fields generated by transmitter 106 may be distorted by the one or more distorting apparatuses 120, where each of the transmitted field is distorted separately. According to Faraday's law, eddy currents in conductive metals are generated by alternating magnetic flux through conductive materials and are proportional to the amplitude and the frequency of the generated alternating magnetic field. Therefore, for low-frequency magnetic fields (for example, lower than 1 kHz, or lower than 500 Hz, or lower than 400 Hz, or lower than 300 Hz, or lower than 200 Hz, or lower than 100 Hz), the generated eddy currents and their corresponding distortion fields drop in correlation with the frequencies. In the case of low-frequency generated magnetic fields and DC electromagnetic sensors (which are unaffected by the low transmission frequency), eddy currents are small, and the dominant magnetic distortion is caused by magnetization of magnetically permeable components, such as paramagnetic or diamagnetic materials. In this case, the distortion response is practically the same for magnetic fields that differ in frequencies (since the distortion is caused by magnetization of metals rather than by generated eddy currents which are frequency dependent). Therefore, in this case, the distortion model 130 may be simplified, e.g. model the distortion fields as one or more dipole fields, with a direction corresponding to the directions of the generated magnetic field, for example aligned or opposite to the generated magnetic fields at the one or more dipole locations. This is true since the magnetization occurs in response to the generated magnetic fields, which are known in space.

In some embodiments, the type of magnetization depends on the type of the magnetized material. Some example types of magnetizations are ferromagnetism, diamagnetism, paramagnetism.

### Exemplary Use of the Sensors 124 to Optimize the Tracking

In some embodiments, having the sensors 124 attached to the body of the patient enables monitoring of the motion and/or momentary positions of the body. In some embodiments, optionally, the sensors 124 include motion sensors, such as accelerometer and/or gyroscope, which may potentially improve the ability of processing module 104 to calculate accurate motion of the body, for example by minimizing calculation errors due to a distorting apparatus 120. For example, distorting apparatus 120 may include a moving imaging device such as a fluoroscopic C-arm, which may distort the magnetic fields dynamically and cause sensing of a false motion by sensors 124. Including position parameters of sensors 124 in the error function together with motion sensor parameters, may enable minimization of such errors.

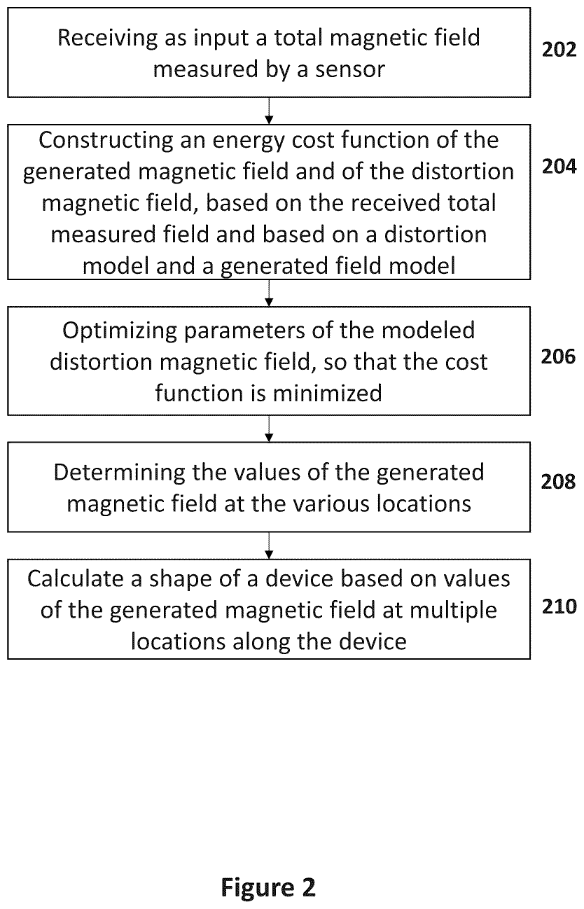

Referring now to FIG. 2, showing a flowchart of an exemplary method 200 for shielding an in-vivo interventional system from an external magnetic distortion, according to some embodiments of the invention.

In some embodiments, the method for shielding an in-vivo interventional system from an external magnetic distortion comprises one or more of the following actions:
1. Receiving as input a total magnetic field values measured by corresponding various sensors 124 and/or 112 (202).

In some embodiments, the measurements of separate magnetic fields may be the result of demultiplexing single magnetic field sensing over time into sensed magnetic fields at multiple frequencies, as described above.

In some embodiments, the total magnetic field \vec{B}ₜₒₜ measured by a sensor 124 or a sensor 112 is a sum of the generated magnetic field \vec{B}_{TX}, generated by transmitter 106, and the distorting magnetic field \vec{B}_{dist}, generated by the one or more distorting apparatuses 120:

$${\vec{B}}_{tot} ( {\{ \vec{r} , q \}}_{0} ) = {\vec{B}}_{TX} ( {\{ \vec{r} , q \}}_{0} ) + {\vec{B}}_{dist} ( {\{ \vec{r} , q \}}_{0} )$$

wherein {\vec{r},q}₀ are a 3D position and a quaternion, which can also be thought of as 6-DOF position and orientation of the measuring sensor. In some embodiments, the processor 104 comprises instructions to calculate a location of a sensor based on the generated magnetic field \vec{B}_{TX}, according to the equation:

$${\{ \vec{r} , q \}}_{0} = \{ \vec{r} , q \} ( {\vec{B}}_{TX} ) ,$$

However, in order to extract the generated magnetic field \vec{B}_{TX} from the total field \vec{B}ₜₒₜ({\vec{r},q}₀), the distorting field component \vec{B}_{dist} needs to be determined, as will be disclosed below.
2. Constructing an energy cost function of the generated magnetic field and the distortion magnetic field possibly generated by the one or more distorting apparatuses 120, based on (a) the received total measured field and (b) a distortion model 130 and/or a generated field model 132 (204).
3. Optimizing parameters of the modeled distortion magnetic field (for example magnitude value, position and orientation of the modeled dipole) so that the cost function is minimized (206).

In some embodiments, the optimization is made under known geometrical constraints such as relative positions and orientations of the various sensors or such as smoothness and/or length constraints.

In some embodiments, the optimization is performed by determining modeled parameter values of one or more of: a. the shield sensors 124; b. the elongated interventional device sensors 112; and c. the distortion magnetic fields; so that the energy cost function is minimized.

Thus, for example, the processing module 104 comprises instructions for determining accurate magnetic field values with modeled distortion field values at sensors 124 and/or 112 and/or at various locations along the interventional device 102, and calculate an accurate shape, positions, and/or orientations along the interventional device 102 unaffected by the distortion fields.

In some embodiments, some of the parameters of distortion model 130 are known to the processing module 104 in advance and can be used to accelerate the optimization and/or make it more precise by eliminating some of the degrees of freedom or adding further constraints. For example, a distance, or a range of possible distances, or a location (or even only the distance along one axis) of the distortion shield 108 with respect to the transmitter 106 may be known, for example based on the distortion shield 108 being attached to the patient's body or installed on a holder of known size and location.

Additionally, for example, a distance, or a range of possible distances, or a location (or even only the distance along one axis) of the distorting apparatus 120 with respect to the transmitter 106 may be known, exactly or approximately. For example, the distance can be known if the distorting apparatus 120 is a known device with known location, position and/or sizes, such as a CT, CBCT (Cone-beam CT) or C-arm in an operation setting. Or, for example, in case the patient is placed on a table/bed covered with metal plates, the metal plates being the distorting apparatus 120.

Additionally, for example, the positioning of the distorting apparatus 120 with respect to the distortion shield 108 may be known to the processing module 104. In some embodiments, the distorting apparatus 120 is a pacemaker or some other device attached to a surface of the patient's body. In this case, the absolute temporal positions of the surface, of the distortion shield 108 and of the distorting apparatus 120 are unknown, however, their relative position may be constrained. In some embodiments, the distorting apparatus 120 is attached to the distortion shield 108 or is part of the distortion shield 108. In such cases the distortion may be strong, however the location of the distorting apparatus 1202 with respect to the distortion shield 108 is known with a very high certainty.
4. Determining the values of the generated magnetic field at the various locations along the interventional device 102 and/or on the distortion shield 108 (208).
5. Calculating various positions and orientations on the distortion shield 108 corresponding to various of the determined magnetic field values, and/or calculating a shape, positions, and/or orientations along the interventional device 102 that correspond to various of the determined magnetic field values (210).

For example, the processing module 104 comprises instructions to calculate a shape and/or position of the interventional device 102 based on values of the generated magnetic field at multiple locations and/or sensors 112 along the interventional device 102. For example, the positions and orientations of the distortion shield magnetic sensors 124 are solved to determine and track, for example, dynamic, positions and orientations of the distortion shield grid.

In some embodiments, the tracked positions and orientations of the distortion shield grid sensors 124 are used to track a surface of the patient's body.

In some embodiments, the tracked surface of the patient's body is used in the procedure to model the patient's movements, for example, to update a real-time registration between tracked tools and the patient's anatomy.

In some embodiments, the tracked surface of the patient's body is used to track the deformation in a deformable registration model of the patient's anatomy. For example, a deformation model may be used that models the deformation of an organ based on the deformation of the tracked patient's surface. For example, the deformation of the lung or of the liver can be modeled by the deformation of the patient's chest (on which the tracked distortion shield may be attached). For example, the deformation model uses interpolation or extrapolation methods to predict the deformation inside the organ by tracking the deformation of the patient's surface (for example, using the tracked distortion shield). For example, the tracked distortion shield surface can be used to detect patient's breathing phase and amplitude, patient's posture or change in posture, patient's body movement, patient's coughs, or any other conditions or events which may affect the deformation of the organ which is being operated, or which may have clinical significance for the physician during the procedure.

In some embodiments, the tracked surface of the patient's body is used to track the breathing and the deformation caused by the breathing to the patient's anatomy. In some embodiments, the tracked surface of the patient's body is used to provide registration between the patient and a preoperative scan of the patient, for example, by segmenting the patient's surface in the preoperative scan and matching it to the real-time tracked surface of the patient's body.

In some embodiments, the tracked surface of the patient's body is used to improve shape and localization calculations by incorporating in the calculation the patient's movement with respect to the transmitter.

In some embodiments, the sensor grid may measure, for example, the patient's temperature, heart rate, ECG, perspiration, respiration, and/or any other suitable physiological parameters.

### Exemplary Method Using Just the Shield Sensors to Compensate for Distortion:

In some embodiments, an exemplary method to compensate for EM distorting fields comprises one or more of the following actions:
1. Under the following assumptions:
a. Under 3 system generated EM fields, assume that the distortion field can be modeled, for example as 3 magnetic dipoles with a certain 3D position (x, y, z) in space, and amplitudes (Dₓ, D_{y}, D_{z}) (total 6 distortion parameters to be searched, referred hereinafter as DIST6). Each generated field may correspond to a single distortion dipole in space, with a certain position, direction and strength. Under the assumption of a ferromagnetic distortion, all distortion dipoles (which correspond to all generated fields, for example, to 3 generated fields) can be assumed to share the same 3D position in space. Additionally or alternatively, each distortion dipole can be assumed to be generated in response to magnetization of a ferromagnetic material along the direction of the system's generated field at the location of the distortion dipole, such that rather than parametrizing each distortion dipole's direction, the dipole's direction may be estimated using the dipole's position and the system's generated field direction and strength at that position in space (according to the system's known generated fields model). It may thus be sufficient to parametrize a distorter using just 6 parameters, DIST6, as mentioned above;
b. Assume that the N shield sensors read the following magnetic field measurements (which are a superposition of both the TX generated fields and the distortion fields): X₁, X₂, . . . , X_{N};
c. Assume that the shield sensors are located at unknown positions and orientations in 3D space: {r,q}₁, {r,q}₂, . . . , {r,q}N (each being a 3D position and a quaternion, each can also be thought of as 6-DOF position and orientation);

### Constructing an Energy Cost Function:

$$E ( x ) = E ( D IST 6 , {\{ r , q \}}_{1} , {\{ r , q \}}_{2} , … , {\{ r , q \}}_{N} ) = {\sum }_{i = 1}^{N} {‖ {B}_{T X} ( {\{ r , q \}}_{i} ) + {B}_{d i s t} ( {\{ r , q \}}_{i} , DIST 6 ) - {X}_{i} ‖}^{2}$$

That is, the sum of all errors between the total predicted field (B_{TX}+B_{dist}) which is TX generated+distortion modeled, and the actual measured fieldsXᵢ. If the true shield sensor 6-DOF locations {r,q}ᵢ were found, and the true distortion parameters DIST6 were found, then all errors are expected to be 0 and E(x) to be minimized. In some embodiments, the “x” in E(x) is the full state vector containing all the unknowns, including the position and orientations of all sensors and the parameters in the distortion model.
2. Finding the optimal parameters for E(x) by minimizing E(x) using non-linear iterative optimization methods;
3. If E(x) is minimized, this means that the shield sensor locations {r,q}ᵢ have been found, and DIST6—the distortion parameters—have been found.

In some embodiments, at this point the distortion fields can be predicted at any location in 3D space.
4. Solving the interventional device's M sensor locations, which includes assuming again that the interventional device sensor measurements are Y₁, Y₂, . . . , Y_{M} and that its unknown 6-DOF locations {R,Q}₁, {R,Q}₂, . . . {R,Q}_{M} are being searched.
5. Constructing an energy cost function:

$$E ( x ) = E ( {\{ R , Q \}}_{1} , {\{ R , Q \}}_{2} , … {\{ R , Q \}}_{M} ) = {\sum }_{i = 1}^{M} {‖ {B}_{T X} ( {\{ R , Q \}}_{i} ) + {B}_{d i s t} ( {\{ R , Q \}}_{i} , DIST 6 ) - {Y}_{i} ‖}^{2}$$

It should be noted that now DIST6 is given and there is no need to search for it (it was already found in (3) using the shield sensors). In this case, DIST6 is used to model the total field B_{TX}+B_{dist}, for solving the interventional device sensor locations.
6. Finding the optimal parameters for E(x) by minimizing E(x) using non-linear iterative optimization methods.
7. If E(x) is minimized, this means that the interventional device sensor locations {R,Q}ᵢ have been found using the distortion modeling.

In this example, the distortion model is first fitted on the measurements of the shield sensors, and then the fitted distortion model is used to solve the interventional device sensor locations.

In some embodiments, this is done by using energy minimization, which was used in order to fit the generated fields+distortion models on the measured data—first on the shield sensor measurements (to fit the distortion model), then on the interventional device sensors.

### Exemplary Method Using Both the Shield Sensors and the Interventional Device Sensors to Both Fit the Distortion Model as Well as Solving all Sensors' Locations at Once:

In some embodiments, an exemplary method for compensate for EM distorting fields comprises one or more of the following actions:
1. Under the following assumptions:
a. Under 3 system-generated EM fields, assume that the distortion field can be modeled, for example as 3 magnetic dipoles with a certain 3D position (x, y, z) in space, and amplitudes (Dₓ, D_{y}, D_{z}) (total 6 distortion parameters to be searched, referred hereinafter as DIST6), as explained above;
b. Assume that the N shield sensors read the following magnetic field measurements (which are a superposition of both the TX generated fields and the distortion fields): X₁, X₂, . . . , X_{N};
c. Assume that the shield sensors are located at unknown positions and orientations in 3D space: {r,q}₁, {r,q}₂, . . . {r,q}N (each being a 3D position and a quaternion, each can also be thought of as 6-DOF position and orientation);
d. Assume that the M interventional device sensors read the following magnetic field measurements (which are a superposition of both the TX generated fields and the distortion fields): Y₁, Y₂, . . . , Y_{M};
e. Assume that the interventional device sensors are located at unknown positions and orientations {R,Q}₁, {R,Q}₂, . . . {R,Q}_{M} (each being a 3D position and a quaternion, each can also be thought of as 6-DOF position and orientation);

### Constructing an Energy Cost Function:

$$E ( x ) = E ( DIST 6 , {\{ r , q \}}_{1} , {\{ r , q \}}_{2} , … {\{ r , q \}}_{N} , {\{ R , Q \}}_{1} , {\{ R , Q \}}_{2} , … {\{ R , Q \}}_{M} ) = {\sum }_{i = 1}^{M} {‖ {B}_{T X} ( {\{ r , q \}}_{i} ) + {B}_{d i s t} ( {\{ r , q \}}_{i} , DIST 6 ) - {X}_{i} ‖}^{2} + {\sum }_{j = 1}^{N} {‖ {B}_{T X} ( {\{ R , Q \}}_{j} ) + {B}_{d i s t} ( {\{ R , Q \}}_{j} , DIST 6 ) - {Y}_{j} ‖}^{2} .$$

That is, the sum of all errors between the total predicted field (B_{TX}+B_{dist}), which is TX-generated+distortion-modeled, and the actual measured fields Xᵢ, Yⱼ for both the shield sensors as well as the interventional device sensors.

If the true sensor 6-DOF locations {r,q}ᵢ, {R,Q}ⱼ (for all values of i and j) were found, and the true distortion parameters DIST6 were found, then all errors are expected to be close to 0 and E(x) to be minimized.
2. Finding the optimal parameters for E(x) by minimizing E(x) using non-linear iterative optimization methods;
3. If E(x) is minimized, this means that the sensor locations {r,q}ᵢ, {R,Q}₁ have been found, as well as DIST6—the distortion parameters—have been found.

In some embodiments, at this point there is no need to continue, since the interventional sensor locations have been found, alongside with the shield sensor locations and the distortion model parameters DIST6.

In some embodiments, by adding more shield sensors into the energy function, more constraints on the DIST6 model (which should fit *all* sensors) can be added, which provides a better optimization.

In some embodiments, the optimization can be performed without using any sensors from the shield, and just by using the interventional device sensors. This may be sufficient to find both the interventional device sensor locations while also fitting the distortion model parameters, which improves the error for the interventional device sensor locations.

In some embodiments, adding smooth/length/shape constraints into the optimization (for example, as Eₛₘₒₒₜₕ=sum of, optionally weighted, 2nd derivatives between {R,Q}ⱼ along elongated device) also improves the optimization, by adding constraints on the solved sensor location and thus effectively reducing the degrees of freedom.

In some embodiments the distortion fields can be modeled using any other model (not necessarily as a set of magnetic dipoles which share the same 3D position in space, and whose directions are determined by their position in space according to the system's generated fields model). In this case, instead of using the DIST6 parameterization, any general parameterization of any general distortion model may be used, for example, “DIST” parameters, which may consist of K parameters, and which may be incorporated in the optimization process above, similarly to how DIST6 was incorporated, as long as B_{dist}({r,q}, DIST) is provided, which models the distortion fields at location {r,q}according to general distortion parameters DIST. This enables using any kind of distortion modeling B_{dist} which may consist of any number of distortion parameters DIST, as long as the number of total searched parameters is smaller than or equal to the sum of measurements and constraints, such that the system of equations is solvable, if not overdetermined.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A system for shielding an in-vivo interventional EM tracking system from an external magnetic distortion, the system comprising:
a. a plurality of magnetic field sensors, aligned in relative positions and orientations with known shape constraints;
b. a processing module;
c. a memory storing:
i. a distortion model of a distorting magnetic field produced by one or more distorting apparatuses; and
ii. a generated field model modeling the magnetic field generated by a transmitter; and
iii. instructions to instruct said processing module for:
A. receiving total magnetic field values measured by corresponding sensors from said plurality of magnetic field sensors; and
B. determining modeled relative positions and orientations with said known shape constraints of said plurality of magnetic field sensors and parameters values of the distortion magnetic field by fitting said distortion model and said generated field model to said received total magnetic field values.

2. The system according to claim 1, wherein said instructions further comprise constructing an energy cost function of the generated field and the distortion magnetic field generated by the one or more distorting apparatuses; and wherein said fitting is so that the energy cost function is minimized.

3. The system according to claim 1, wherein said plurality of magnetic field sensors are mounted on at least one distortion shield; and
wherein said at least one distortion shield is configured to be disposed between or in the proximity of a magnetic field transmitter and one or more distorting apparatuses.

4. (canceled)

5. The system according to claim 1, wherein said plurality of magnetic field sensors are mounted along at least one interventional elongated device.

6. The system according to claim 2, wherein said constructing an energy cost function is based on:
a. said received total measured fields;
b. said distortion model; and
c. said generated field model.

7. The system according to claim 1, wherein said distortion model is based on one or more of:
a. approximating said distorting magnetic fields as magnetic fields produced by one or more magnetic dipoles;
b. approximating said distorting magnetic fields as magnetic fields produced by one or more electrical current loops; and
c. approximated magnetic permeability properties of said one or more distorting apparatuses.

8-9. (canceled)

10. The system according to claim 3, wherein said plurality of magnetic field sensors of said on at least one distortion shield are one or more of:
a. fixed to a body of a patient; and wherein the processing module is configured to calculate motion parameter values of said body based on said measured magnetic fields;
b. combined with motion sensors for sensing motion of a body of a patient.

11. The system according to claim 2, wherein said energy cost function is further constructed based on modeled motion parameters that are determined so that said energy cost function is minimized.

12. (canceled)

13. The system according to claim 1, wherein said determining modeled parameter values of said plurality of magnetic field sensors is made with relative positions and orientations of said plurality of magnetic field sensors as constraints.

14. The system according to claim 2, further comprising at least one interventional elongated device; and wherein said energy function comprises modeled parameters of said at least one interventional elongated device; and wherein one or more of the following is true:
a. said energy function comprises smoothness and/or length constraints of said at least one interventional elongated device;
b. said energy function comprises sensed values of magnetic fields at multiple sensors along said at least one interventional elongated device; and wherein said instructions further comprise determining parameter values of said plurality of magnetic field sensors of said at least one interventional elongated device so that the cost function is minimized;
c. said instructions further comprise determining positions and orientations of said plurality of magnetic field sensors of said at least one interventional elongated device based on optimization approximated sensed values of magnetic fields;
d. said instructions further comprise receiving total magnetic field values measured by corresponding various sensors from said plurality of magnetic field sensors of said at least one interventional elongated device; wherein said instructions further comprise constructing said energy cost function further based on modeled parameters of said plurality of magnetic field sensors of said at least one interventional elongated device; wherein said instructions further comprise determining parameter values of said plurality of magnetic field sensors of said at least one interventional elongated device, so that the energy cost function is minimized; and
e. said instructions further comprise determining a shape of said at least one interventional elongated device, based on optimization approximated sensed values of magnetic field.

15-21. (canceled)

22. The system according to claim 5, further comprising displaying a shape and/or position of said at least one interventional elongated device based on said determining.

23. The system according to claim 3, said instructions further comprise determining positions and orientations of said plurality of magnetic field sensors of said on at least one distortion shield based on sensed values of magnetic fields; and wherein:
a. said positions and orientations of said plurality of magnetic field sensors of said on at least one distortion shield are solved to determine and track dynamic positions and orientations of said at least one distortion shield;
b. said tracked positions and orientations of said plurality of magnetic field sensors of said on at least one distortion shield are used to track a surface of a body of a patient; and
wherein said tracked surface is used for one or more of:
i. to model movements of said patient; said modeled movements are used to update a real-time registration between tracked tools and an anatomy of said patient;
ii. to track a deformation in a deformable registration model of an anatomy of said patient;
iii. to track one or more of a breathing phase, an amplitude and a deformation caused by breathing to an anatomy of said patient;
iv. to provide registration between said patient and a preoperative scan of said patient by segmenting a surface in said preoperative scan and matching said segmented surface to said tracked surface;
v. to improve shape and localization calculations by incorporating in said calculations movement of said patient with respect to a transmitter.

24. (canceled)

25. The system according to claim 3, said at least one distortion shield is further configured for measuring at least one of temperature, heart rate, ECG, perspiration and respiration of a patient.

26. A method for shielding an in-vivo interventional EM tracking system from an external magnetic distortion, comprising:
a. receiving total magnetic field values measured by corresponding sensors from a plurality of magnetic field sensors; and
b. determining modeled parameter values of said plurality of magnetic field sensors and of the distortion magnetic field by fitting said distortion model and said generated field model to said received total magnetic field values.

27. The method according to claim 26, further comprising constructing an energy cost function of the generated field and the distortion magnetic field generated by one or more distorting apparatuses; and wherein said fitting is so that the energy cost function is minimized.

28. The method according to claim 26, further comprising mounting said plurality of magnetic field sensors on one or more of:
a. at least one distortion shield; further comprising disposing said at least one distortion shield between or in the proximity of a magnetic field transmitter and one or more distorting apparatuses;
b. along at least one interventional elongated device.

29-30. (canceled)

31. The method according to claim 27, further comprising basing said constructing an energy cost function on:
a. said received total measured fields;
b. said distortion model; and
c. said generated field model.

32. The method according to claim 26, further comprising basing said distortion model on one or more of:
a. approximating said distorting magnetic fields as magnetic fields produced by one or more magnetic dipoles;
b. approximating said distorting magnetic fields as magnetic fields produced by one or more electrical current loops;
c. approximated magnetic permeability properties of said one or more distorting apparatuses.

33-34. (canceled)

35. The method according to claim 28, further comprising fixing said plurality of magnetic field sensors of said on at least one distortion shield to a body of a patient; and further comprising calculating motion parameter values of said body based on said measured magnetic fields.

36. The method according to claim 27, further comprising further constructing said energy cost function based on modeled motion parameters; and further comprising further determining said modeled parameter values so that said energy cost function is minimized.

37. The method according to claim 28, further comprising combining said plurality of magnetic field sensors of said on at least one distortion shield with motion sensors for sensing motion of a body of a patient.

38. The method according to claim 26, wherein said determining modeled parameter values of said plurality of magnetic field sensors is made with relative positions and orientations of said plurality of magnetic field sensors as constraints.

39. The method according to claim 27, wherein said energy function comprises one or more of:
a. modeled parameters of at least one interventional elongated device;
b. smoothness and/or length constraints of said at least one interventional elongated device;
c. sensed values of magnetic fields at multiple sensors along said at least one interventional elongated device; and further comprising determining parameter values of said plurality of magnetic field sensors of said at least one interventional elongated device so that the cost function is minimized.

40-41. (canceled)

42. The method according to claim 39, further comprising one or more of:
a. determining positions and orientations of said plurality of magnetic field sensors of said at least one interventional elongated device based on optimization approximated sensed values of magnetic fields;
b. receiving total magnetic field values measured by corresponding various sensors from said plurality of magnetic field sensors of said at least one interventional elongated device; further comprising constructing said energy cost function further based on modeled parameters of said plurality of magnetic field sensors of said at least one interventional elongated device; further comprising determining parameter values of said plurality of magnetic field sensors of said at least one interventional elongated device, so that the energy cost function is minimized; and
c. determining a shape of said at least one interventional elongated device, based on optimization approximated sensed values of magnetic field.

43-46. (canceled)

47. The method according to claim 30, further comprising displaying a shape and/or position of said at least one interventional elongated device based on said determining.

48. The method according to claim 28, further comprising determining positions and orientations of said plurality of magnetic field sensors of said on at least one distortion shield based on sensed values of magnetic fields;
further comprising solving said positions and orientations of said plurality of magnetic field sensors of said on at least one distortion shield for determining and tracking dynamic positions and orientations of said at least one distortion shield; and
further comprising one or more of:
a. using said tracked positions and orientations of said plurality of magnetic field sensors of said on at least one distortion shield for tracking a surface of a body of a patient;
b. using said tracked surface for modeling movements of said patient;
c. using said modeled movements for updating a real-time registration between tracked tools and an anatomy of said patient;
d. using said tracked surface for tracking a deformation in a deformable registration model of an anatomy of said patient;
e. using said tracked surface for tracking one or more of a breathing phase, an amplitude and a deformation caused by breathing to an anatomy of said patient;
f. using said tracked surface for providing registration between said patient and a preoperative scan of said patient;
g. segmenting a surface in said preoperative scan and matching said segmented surface to said tracked surface;
h. using said tracked surface for improving shape and localization calculations by incorporating in said calculations movement of said patient with respect to a transmitter;
i. measuring at least one of temperature, heart rate, ECG, perspiration and respiration of a patient using said at least one distortion shield.

49-50. (canceled)
